(19) United States
(12) Patent Application Publication
(10) Pub. No.: US 2012/0208855 A1
(43) Pub. Date: Aug. 16, 2012

(54) AMINO-BENZOIC ACID DERIVATIVES FOR USE IN THE TREATMENT OF DIHYDROGENASE-RELATED DISORDERS

(75) Inventors: Larry Raymond Mclean, Bridgewater, NJ (US); Arun Subramaniam, Lebanon, NJ (US); Roy J. Vaz, Bridgewater, NJ (US); Yi Li, Bridgewater, NJ (US)

(73) Assignee: SANOFI, Paris (FR)

(21) Appl. No.: 13/504,039

(22) PCT Filed: Oct. 15, 2010
(86) PCT No.: PCT/US10/52768

§ 371 Date: Apr. 25, 2012

(30) Foreign Application Priority Data
Jul. 12, 2010 (FR) 1055646

(51) Int. Cl. C12N 9/99; A61K 31/341; A61P 19/02; A61P 37/00; A61P 35/04; A61K 31/195; A61K 31/4406

(52) U.S. Cl. 514/357; 435/184; 514/564; 514/471

(57) ABSTRACT

Provided herein is method of modulating, and in particular decreasing dihydroorotate dehydrogenase (DHODH) activity, as well as a method of treating a DHODH related disease or disorder.

### FIELD OF THE INVENTION

The present invention generally relates to method of modulating the activity of dihydroorotate dehydrogenase as well as a method for treating a dihydroorotate dehydrogenase related disease or disorder.

### BACKGROUND OF THE INVENTION

Dihydroorotate dehydrogenase (DHODH) is an enzyme essential to pyrimidine de novo biosynthesis.¹ It catalyzes oxidative conversion of dihydroorotate to orotate using the co-factors flavin mononucleotide (FMN) and ubiquinone (CoQ) in the redox process. Since blocking pyrimidine biosynthesis has an antiproliferative effect on rapidly dividing cells,² inhibitors of human DHODH (hDHODH) have been pursued and developed for the treatment of cancer,^{3,4} and immunological disorders, such as rheumatoid arthritis⁵ and multiple sclerosis.⁶⁻⁸ The DHODH enzymes of parasitic pathogens such as P. falciparum (PfDHODH) and Trypanosoma brucei are also attractive targets for the development of new therapeutics to combat malaria and sleeping sickness.⁹⁻¹¹

Except for substrate-based inhibitors of pyrimidine analogs,¹² there have been many reports of inhibitors aimed at the putative binding site of the ubiquinone cofactor.¹³⁻³⁰ X-ray crystallographic studies of inhibitor complexes with hDHODH and PfDHODH have revealed that known inhibitors such as brequinar⁴ and teriflunomide (the active metabolite of leflunomide)³¹ abolish the enzyme activity by displacing the ubiquinone co-factor,^{13-16, 32-34} either competitively or otherwise.³⁵ Analogs of brequinar and teriflunomide all contain an acidic head group that interacts with the guanidinyl group of Arg-136 of hDHODH, or the corresponding Arg-265 of PfDHODH, at the ubiquinone binding site. An alternative binding mode wherein the acidic group interacts with Tyr-356 has been reported, with some compounds adopting a dual binding mode.¹⁴

This dual binding mode has been observed in a species dependent manner, where the hydroxyl group of teriflunomide interacts with Arg in hDHODH but with Tyr-528 in PfiDHODH.³³ Compounds with a neutral polar head group have also been reported as DHODH inhibitors^{29, 30}, including the recently disclosed triazolopyrimidine derivatives^{18, 20}, and S-2678.²¹ The X-ray structures showed that the triazolopyrimidine head group acts as hydrogen-bond acceptor interacting with the guanidinyl group of Arg-265.¹⁷

### SUMMARY OF THE INVENTION

There is provided, in accordance with the present invention, a method of modulating activity of DHODH with compounds that surprisingly and unexpectedly, have the ability to modulate, and in particular decrease the activity of DHODH.

Broadly, the present invention extends to A method for modulating dihydroorotate dehydrogenase (DHODH) activity, comprising contacting the DHODH with a compound:

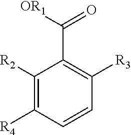

wherein:
R₁ is H, an alkyl or lower alkyl group, or a halogen;

### R₂ is:

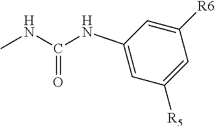

wherein R₅ is a halogen and R₆ is a halogen,

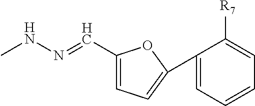

wherein R₇ is a halogen, or

### H;

### R₃ is:

### H,

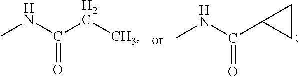

and

### R₄ is:

### H, or

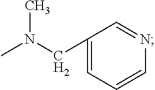

an N-oxide of the compound, a prodrug of the compound, a pharmaceutically acceptable salt of the compound, a solvate of the compound, or a hydrate of the compound.

In a particular embodiment, contacting a compound as described herein with DHODH decreases the DHODH activity with respect to the DHODH activity prior to contacting the compound with the DHODH. Particular examples of a compound having applications in a method of the present invention in which DHODH activity is decreased include, but certainly are not limited to:

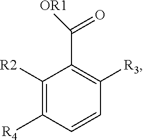

wherein:

### R₁ is H,

### R₂ is

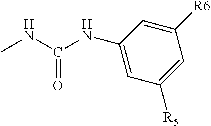

wherein R₅ and R₆ are each a halogen, and in a particular embodiment, are each Cl,

### R₃ is H, and

### R₄ is H;

### R₁ is H,

### R₂ is

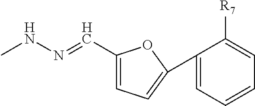

wherein R₇ is Cl,

### R₃ is H, and

### R₄ is H:

### R₁ is H;

### R₂ is H;

### R₃ is

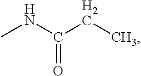

and

### R₄ is

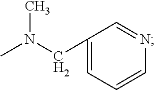

and

### R₁ is H,

### R₂ is H,

### R₃ is

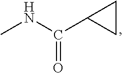

and

### R₄ is

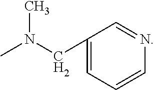

In addition, the present invention extends to a method for decreasing DHODH activity comprising contacting the DHODH with a compound:

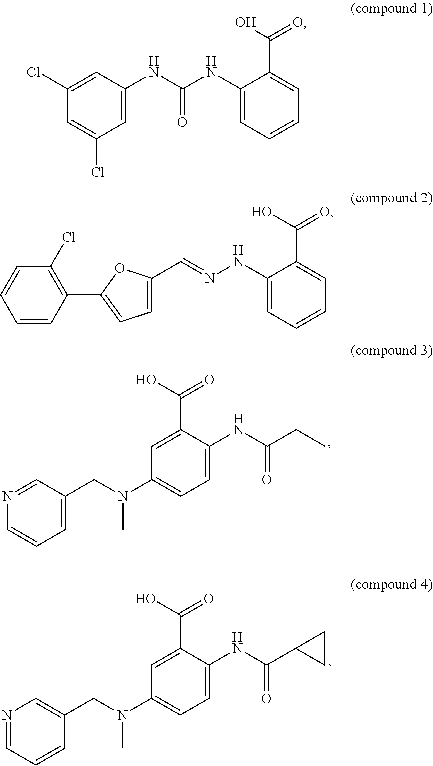

or any combination thereof;
an N-oxide of the compound, a prodrug of the compound, a pharmaceutically acceptable salt of the compound, a solvate of the compound, or a hydrate of the compound, wherein contacting the compound with the DHODH decreases the DHODH activity with respect to the DHODH activity prior to contacting the compound with the DHODH.

Moreover, the present invention extends to a pharmaceutical composition comprising a compound and a pharmaceutically acceptable carrier, wherein the compound is wherein the compound is:

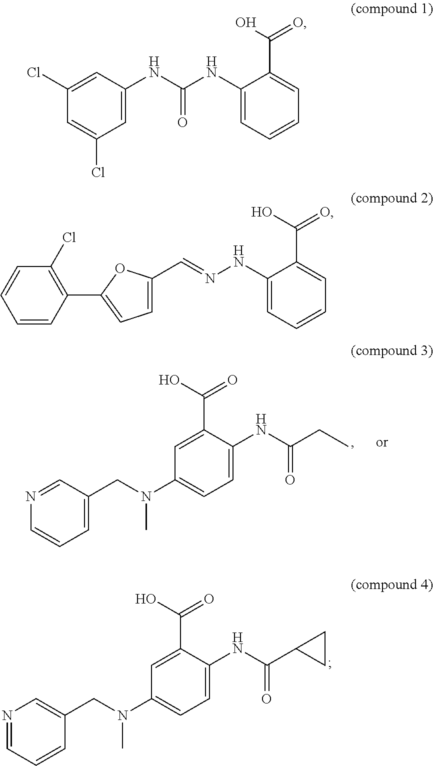

an N-oxide of the compound, a prodrug of the compound, a pharmaceutically acceptable salt of the compound, a solvate of the compound, or a hydrate of the compound.

In addition, the present invention extends to a method of preventing, treating or ameliorating a DHODH related disease or disorder in a subject, comprising administrating to the subject a therapeutically effective amount of a compound:

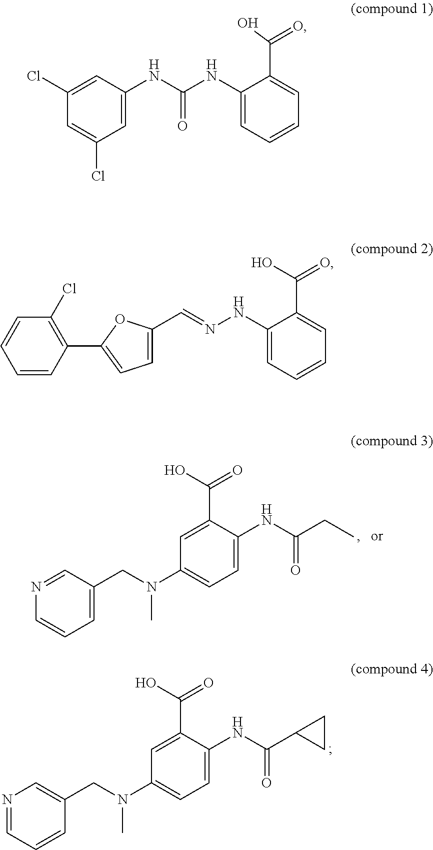

an N-oxide of the compound, a prodrug of the compound, a pharmaceutically acceptable salt of the compound, a solvate of the compound, or a hydrate of the compound.

Numerous types of DHODH related diseases or disorders can be prevented, treated, or ameliorated with a method of the present invention. Particular examples of such a disease or disorder include an immunological disorder and cancer, to name only a few. Moreover, examples of an immunological disorder that can prevented, treated or ameliorated with a method of the present invention includes, but certainly is not limited to rheumatoid arthritis and multiple sclerosis.

The present invention further extends to the use of a compound described herein, particularly

Compound 1, 2, 3, or 4 in a medicament for the treatment, prevention or amelioration of a DHODH related disease or disorder.

### BRIEF DESCRIPTION OF THE DRAWINGS

FIG. 1: Binding mode of compound 1 with DHODH revealed by X-ray crystallography.

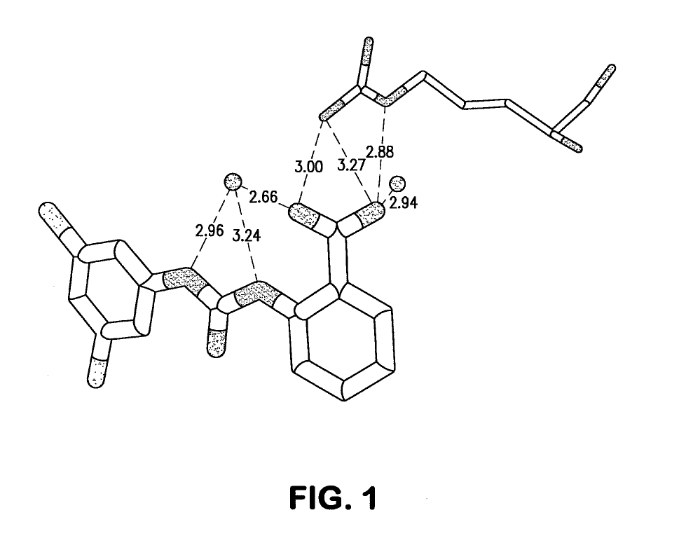

FIG. 2: Binding mode of compound 1 (orange) and 2 (white) with DHODH determined by X-ray crystallography.

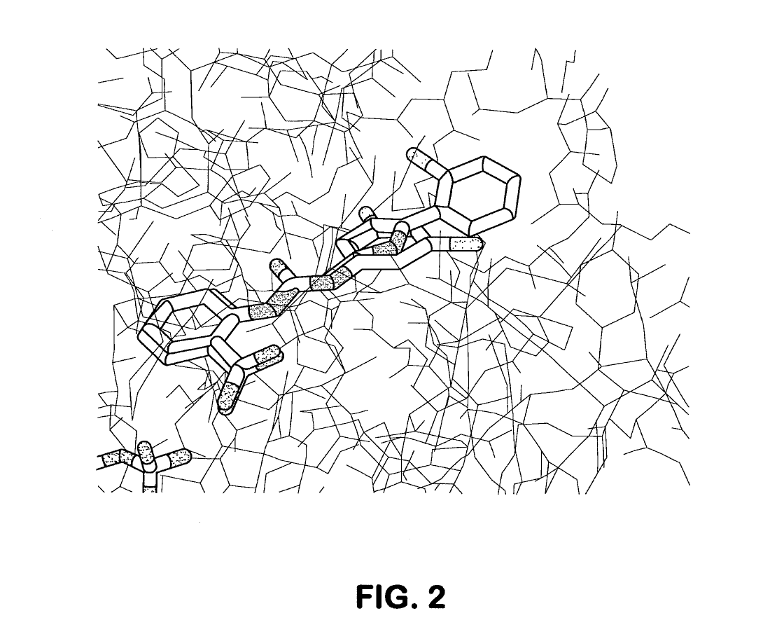

FIG. 3: Binding mode of compound 2 (white) and 3 (orange) with DHODH determined by X-ray crystallography.

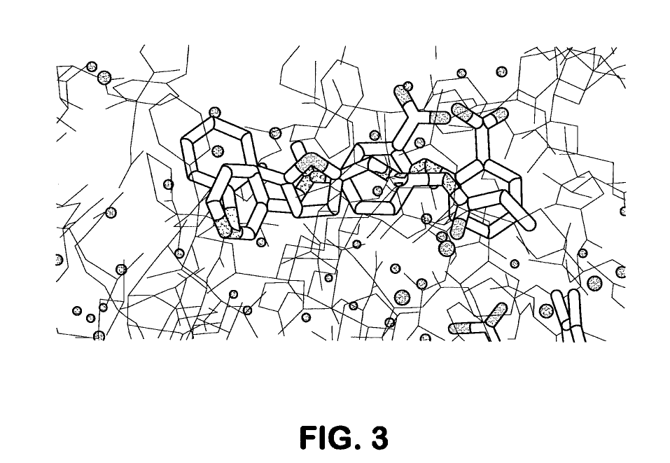

FIG. 4: Superposition of compounds 1&2 (gray wire-frame), 3&4 (orange wire-frame) and inhibitor of 2PRL (gray stick).

### DETAILED DESCRIPTION OF THE INVENTION

The present invention is based on the discovery that surprisingly and unexpectedly, heretofore known compounds have a heretofore unknown ability to modulate, and in particular decrease the activity of DHODH. As explained above, DHODH plays a role in various diseases or disorders, including but certainly not limited to immunological diseases and disorders, such as rheumatoid arthritis and multiple sclerosis, as well as in cancer. Thus administering a compound that modulates, and in particular decreases the activity of DHODH in a subject readily can be used in a method to prevent, treat or ameliorate such a DHODH related disease or disorder in the subject.

Hence broadly, the present invention extends to a method for modulating dihydroorotate dehydrogenase (DHODH) activity, and in particular decreasing DHODH activity, comprising contacting the DHODH with a compound:

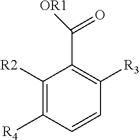

wherein:
R₁ is H, an alkyl or lower alkyl group, or a halogen;

### R₂ is:

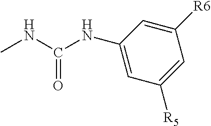

wherein R₅ is a halogen and R₆ is a halogen,

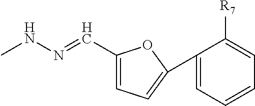

wherein R7 is a halogen, or

### H;

### R₃ is:

### H,

and

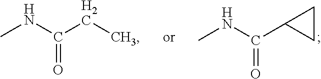

and

### R₄ is:

### H, or

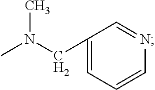

an N-oxide of the compound, a prodrug of the compound, a pharmaceutically acceptable salt of the compound, a solvate of the compound, or a hydrate of the compound.

The present invention further extends to a method of preventing, treating or ameliorating a DHODH related disease or disorder in a subject, comprising administrating to the subject a therapeutically effective amount of a compound:

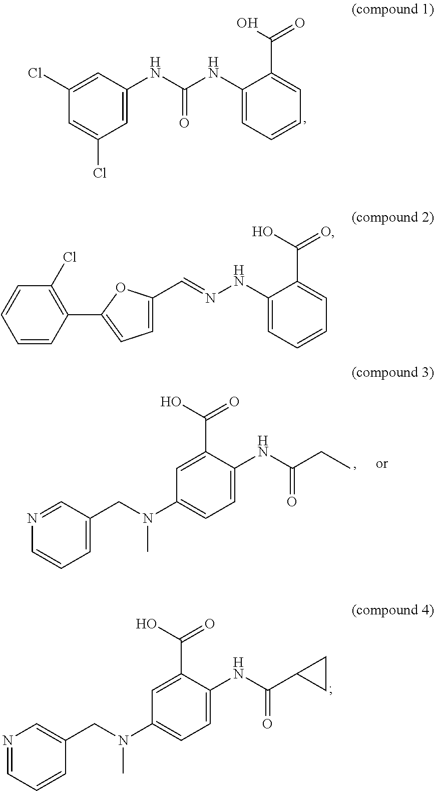

an N-oxide of the compound, a prodrug of the compound, a pharmaceutically acceptable salt of the compound, a solvate of the compound, or a hydrate of the compound.

Numerous terms and phrases used throughout the instant specification and appended claims are defined below.

Accordingly, as used herein the terms “modulating”, “modulate”, “modulation” or any form of the word refers to a change in the activity of DHODH as compared to a control DHODH or as compared to activity of the DHODH prior to contact with a compound as described herein. The change in activity can be an increase in the DHODH activity as compared to activity of the control DHODH or compared to the DHODH activity prior to contact with a compound as described herein, or a decrease in the DHODH activity as compared to activity of the control DHODH or compared to the DHODH activity prior to contact with a compound as described herein.

As used herein, the phrase “decreases DHODH activity” refers to a change in activity of DHODH as compared to a control DHODH or prior to contact with a compound as described herein, wherein upon contact with contact with a compound described herein, the DHODH activity is less than the DHODH activity prior to contact with a compound described herein, or less than the DHODH activity of a control DHODH.

As used herein, the term “alkyl” means, unless otherwise specified, an aliphatic hydrocarbon group which may be straight or branched having about 1 to about 15 carbon atoms in the chain optionally substituted by alkoxy or by one or more halogen atoms. Particular alkyl groups have from 1 to about 6 carbon atoms. “Lower alkyl” as a group or part of a lower alkoxy, lower alkylthio, lower alkylsulfinyl or lower alkylsulfonyl group means unless otherwise specified, an aliphatic hydrocarbon group which may be straight or branched having about 1 to about 4 carbon atoms in the chain. Exemplary alkyl groups include methyl, ethyl, n-propyl, i-propyl, n-butyl, s-butyl, t-butyl, n-pentyl, 3-pentyl, heptyl, octyl, nonyl, decyl and dodecyl.

As used herein, the term “halogen” refers to fluoro (F), chloro (Cl), bromo (Br), or iodo (I).

As used herein, the term “prodrug” refers to a compound which is suitable for administration to a subject without undue toxicity, irritation, allergic response, and the like, and is convertible in vivo by metabolic means (e.g. by hydrolysis) to a compound having applications in a method of the present invention, in particular compound 1, 2, 3, or 4, including N-oxides thereof. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987.

### Pharmaceutical Compositions

As explained above, the present invention extends to a method of treating a DHODH related disease or disorder in a subject, comprising administering to the subject a compound as described herein, and in particular Compound 1, 2, 3, or 4, or a combination thereof. Thus, also provided herein is a pharmaceutical composition comprising a compound as described herein and a pharmaceutically acceptable carrier thereof. As used herein, the term “pharmaceutically acceptable” preferably means approved by a regulatory agency of a government, in particular the Federal government or a state government, or listed in the U.S. Pharmacopeia or another generally recognized pharmacopeia for use in animals, and more particularly in humans. Suitable pharmaceutical carriers are described in “Remington's Pharmaceutical Sciences” by E. W. Martin.

A pharmaceutical composition according to the present invention can be prepared according to the customary methods, using one or more pharmaceutically acceptable adjuvants or excipients. The adjuvants comprise, inter alia, diluents, sterile aqueous media and the various non-toxic organic solvents. A pharmaceutical composition of the present invention may be presented in the form of tablets, pills, granules, powders, aqueous solutions or suspensions, injectable solutions, elixirs or syrups, and can contain one or more agents chosen from the group comprising sweeteners, flavorings, colorings, or stabilizers in order to obtain pharmaceutically acceptable preparations. The choice of vehicle and the content of active substance in the vehicle are generally determined in accordance with the solubility and chemical properties of the compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used. Such pharmaceutically acceptable carriers can also be sterile water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include mannitol, human serum albumin (HSA), starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol, water, ethanol and the like. These compositions can take the form of solutions, suspensions, tablets, pills, capsules, powders, sustained-release formulations and the like.

Naturally, a pharmaceutical composition of the present invention compositions will contain an effective diagnostic or therapeutic amount of the active compound together with a suitable amount of carrier so as to provide the form for proper administration to the subject. While intravenous injection is a very effective form of administration, other modes can be employed, such as by injection, or by oral, nasal or parenteral administration, which are discussed infra.

### Methods of Treatment

Numerous DHODH related disease or disorder can be prevented, treated or ameliorated with a method of the present invention. Examples of such DHODH related diseases or disorders include, but certainly are not limited to inflammatory diseases, for example joint inflammation, including arthritis, rheumatoid arthritis and other arthritic conditions such as rheumatoid spondylitis, gouty arthritis, traumatic arthritis, rubella arthritis, psoriatic arthritis, osteoarthritis and other chronic inflammatory joint diseases, or diseases of joint cartilage destruction, ocular conjunctivitis, vernal conjunctivis, inflammatory bowel disease, asthma, allergic rhinitis, interstitial lung diseases, fibrosis, sceleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina and other consequences of atherosclerotic plaque rupture, as well as periodontal disease, diabetic retinopathy, tumor growth, anaphylaxis, multiple sclerosis, peptic ulcers, and syncytial viral infections.

Cancers are also DHODH related diseases or disorders that can be prevented, treated or ameliorated with a method of the present invention. A cancer that can be treated with a method of the present invention includes solid tumors, such as sarcomas and carcinomas, which include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Furthermore, a cancer involving dysproliferative changes (such as metaplasias and dysplasias) in epithelial tissues such as those in the cervix, esophagus, and lung can also be treated ameliorated or prevented with a method of the present invention.

In addition, a method of the present invention also prevents, treats or ameliorates conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

A method of the present invention can also be used in the treatment, prevention or amelioration of non-malignant tumors and other disorders involving inappropriate cell or tissue growth. For example, it is contemplated that a method of the present invention is useful for the treatment of arteriovenous (AV) malformations, particularly in intracranial sites. A method of the present invention may also be used to prevent, treat or ameliorate psoriasis, a dermatologic condition that is characterized by inflammation and vascular proliferation; benign prostatic hypertrophy, a condition associated with inflammation and possibly vascular proliferation; and cutaneous fungal infections. Treatment, prevention or amelioration of other hyperprobiferative disorders with a method of the present invention is also contemplated.

### Combination Therapy

As explained above, other pharmaceutically active agents can be employed in a method of the present invention with a compound described herein, depending upon the disease or disorder being treated. For example, in the treatment of asthma, beta-adrenergic agonists such as albuterol, terbutaline, formoterol, fenoterol or prenaline can be included, as can anticholinergics such as ipratropium bromide, anti-inflammatory corticosteroids such as beclomethasone dipropionate, triamcinolone acetonide, flunisolide or dexamethasone, and anti-inflammatory agents such as sodium cromoglycate and nedocromil sodium. Thus, the present invention extends to a pharmaceutical composition comprising a compound described herein, and particular compound 1, 2, 3, or 4, and a second compound selected from the group consisting of a beta andrenergic agonist, an anticholinergic, an anti-inflammatory corticosteroid, and an anti-inflammatory agent; and a pharmaceutically acceptable carrier thereof, examples of which are discussed above.

### Modes of Delivery

In a method of the present invention for preventing, treating or ameliorating a DHODH related disease or disorder in a subject, a compound as described herein, and particular compound 1, 2, 3, or 4, or a pharmaceutical composition of the present invention, may be introduced parenterally, transmucosally, e.g., orally, nasally, pulmonarily, or rectally, or transdermally to the subject.

### Oral Delivery

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for a therapeutic is given by Marshall, K. In: Modern Pharmaceutics Edited by G. S. Banker and C. T. Rhodes Chapter 10, 1979, herein incorporated by reference. In general, the formulation will include a compound as described herein, and particular compound 1, 2, 3, or 4, and inert ingredients which allow for protection against the stomach environment, and release of the biologically active material, i.e., a compound as described herein, particularly compound 1, 2, 3, or 4, in the intestine.

Also specifically contemplated are oral dosage forms of a compound described herein, in particular, compound 1, 2, 3, or 4. Such a compound may be chemically modified so that oral delivery is more efficacious. Generally, the chemical modification contemplated is the attachment of at least one moiety to the component molecule itself, where the moiety permits (a) inhibition of proteolysis; and (b) uptake into the blood stream from the stomach or intestine. It also may be desirous to increase overall stability of a compound of a pharmaceutical composition or method of the present invention, and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For a composition or method of the present invention, the location of release of a compound, particularly compound 1, 2, 3, or 4, may be the stomach, the small intestine (the duodenum, the jejunum, or the ileum), or the large intestine. One skilled in the art has available formulations that will not dissolve in the stomach, yet will release the material in the duodenum or elsewhere in the intestine. Preferably, the release will avoid the deleterious effects of the stomach environment, either by protection of the compound, or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings that make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

A compound of a pharmaceutical composition of the present invention can be included in the formulation as fine multi-particulates in the form of granules or pellets of particle size about 1 mm. The formulation of the compound for capsule administration could also be as a powder, lightly compressed plugs or even as tablets. The therapeutic could be prepared by compression.

Colorants and flavoring agents may all be included. For example, a compound of a pharmaceutical composition or method of the present invention may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include, but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants is an insoluble cationic exchange resin. Powdered gums may be used as disintegrants and as binders and these can include powdered gums such as agar, Karaya or tragacanth. Alginic acid and its sodium salt are also useful as disintegrants.

Binders may be used to hold a compound together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of a pharmaceutical composition of the present invention to prevent sticking during the formulation process. Lubricants may be used as a layer between a compound having applications in a method of the present invention, particularly compound 1, 2, 3, or 4 and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of a compound having applications in a pharmaceutical composition or method of the present invention, particularly compound 1, 2, 3 or 4 into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in a pharmaceutical composition of the present invention either alone or as a mixture in different ratios.

Additives which potentially enhance uptake of a compound having applications in a pharmaceutical composition or method of the present invention are, for instance, the fatty acids oleic acid, linoleic acid and linolenic acid.

Controlled release oral formulation may be desirable. A compound having applications in a method or pharmaceutical composition of the present invention, and in particular compound 1, 2, 3, or 4, could be incorporated into an inert matrix which permits release by either diffusion or leaching mechanisms, e.g., gums. Slowly degenerating matrices may also be incorporated into the formulation. Some enteric coatings also have a delayed release effect.

Another form of a controlled release of a compound having applications in a method or pharmaceutical composition of the present invention, and in particular compound 1, 2, 3, or 4, is by a method based on the OROS therapeutic system (Alza Corp.), i.e. the drug is enclosed in a semipermeable membrane which allows water to enter and push drug out through a single small opening due to osmotic effects.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan-coater or in a fluidized bed or by compression coating.

### Pulmonary Delivery

Also contemplated herein is pulmonary delivery of a compound having applications in a method of the present invention, particularly compound 1, 2, 3, or 4, either alone or in a pharmaceutical composition. The compound is delivered to the lungs of a mammal while inhaling and traverses across the lung epithelial lining to the blood stream. Other reports of this include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-γ and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the ULTRAVENT nebulizer, manufactured by Mallinckrodt, Inc., St. Louis, Mo.; the ACORN II nebulizer, manufactured by Marquest Medical Products, Englewood, Colo.; the VENTOLIN metered dose inhaler, manufactured by Glaxo Inc., Research Triangle Park, N.C.; and the SPINHALER powder inhaler, manufactured by Fisons Corp., Bedford, Mass., to name only a few.

All such devices require the use of formulations suitable for the dispensing a compound having applications in a method of the present invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. A compound having application in a method of the present invention may also be prepared in different formulations depending on the type of chemical modification or the type of device employed.

Formulations suitable for use with a nebulizer, either jet or ultrasonic, will typically comprise a compound having applications in a method of the present invention dissolved in water at a concentration of about 0.1 to 25 mg of compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing a compound having applications in a method of the invention suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a compound having applications in a method of the present invention, and may also include a bulking agent, such as lactose, sorbitol, sucrose, or mannitol in amounts which facilitate dispersal of the powder from the device, e.g., 50 to 90% by weight of the formulation. The compound of the present invention should most advantageously be prepared in particulate form with an average particle size of less than 10 mm (or microns), most preferably 0.5 to 5 mm, for most effective delivery to the distal lung.

### Nasal Delivery

Nasal delivery of a compound described herein having applications in a method of the present invention, and in particular compound 1, 2, 3, or 4 is also contemplated. Nasal delivery allows the passage of the compound to the blood stream directly after administering the therapeutic product to the nose, without the necessity for deposition of the product in the lung. Formulations for nasal delivery include those with dextran or cyclodextran.

### Transdermal Delivery

Various and numerous methods are known in the art for transdermal administration of a drug, e.g., via a transdermal patch, have applications in the present invention. Transdermal patches are described in for example, U.S. Pat. No. 5,407,713, issued Apr. 18, 1995 to Rolando et al.; U.S. Pat. No. 5,352,456, issued Oct. 4, 1004 to Fallon et al.; U.S. Pat. No. 5,332,213 issued Aug. 9, 1994 to D'Angelo et al.; U.S. Pat. No. 5,336,168, issued Aug. 9, 1994 to Sibalis; U.S. Pat. No. 5,290,561, issued Mar. 1, 1994 to Farhadieh et al.; U.S. Pat. No. 5,254,346, issued Oct. 19, 1993 to Tucker et al.; U.S. Pat. No. 5,164,189, issued Nov. 17, 1992 to Berger et al.; U.S. Pat. No. 5,163,899, issued Nov. 17, 1992 to Sibalis; U.S. Pat. Nos. 5,088,977 and 5,087,240, both issued Feb. 18, 1992 to Sibalis; U.S. Pat. No. 5,008,110, issued Apr. 16, 1991 to Benecke et al.; and U.S. Pat. No. 4,921,475, issued May 1, 1990 to Sibalis, the disclosure of each of which is incorporated herein by reference in its entirety.

It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989 to Aruga et al., the disclosure of each of which is incorporated herein by reference in its entirety.

### Topical Administration

For topical administration, gels (water or alcohol based), creams or ointments containing a compound having applications in a method of the present invention, and in particular compound I1, 2, 3, or 4 may be used. Such a compound may also be incorporated in a gel or matrix base for application in a patch, which would allow a controlled release of compound through the transdermal barrier.

### Rectal Administration

Solid compositions for rectal administration include suppositories formulated in accordance with known methods and containing at least one compound described herein that has applications in a method of the present invention.

The percentage of active ingredient in a pharmaceutical composition or the present invention may be varied, it being necessary that it should constitute a proportion such that a suitable dosage shall be obtained. Obviously, several unit dosage forms may be administered at about the same time. The dose employed will be determined by the physician, and depends upon the desired therapeutic effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 1, mg/kg body weight per day by intravenous administration.

In each particular case, the doses will be determined in accordance with the factors distinctive to the subject to be treated, such as age, weight, general state of health and other characteristics which can influence the efficacy of the medicinal product.

Furthermore, a compound having applications in a method of the present invention may be administered as frequently as necessary in order to obtain the desired therapeutic effect. Some subjects may respond rapidly to a higher or lower dose and may find much weaker maintenance doses adequate. For other subjects, it may be necessary to have long-term treatments at the rate of 1 to 4 doses per day, in accordance with the physiological requirements of each particular subject.

Generally, the active product may be administered orally 1 to 4 times per day. Of course, for some subjects, it will be necessary to prescribe not more than one or two doses per day.

Naturally, a subject in whom administration of a compound having applications in a method of the present invention is an effective therapeutic regimen is preferably a human, but can be any animal Thus, as can be readily appreciated by one of ordinary skill in the art, a method and pharmaceutical composition of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

Moreover, as used herein, the term “therapeutically effective” is used herein to mean an amount sufficient to prevent, and preferably reduce by at least about 30 percent, more preferably by at least 50 percent, most preferably by at least 90 percent, a DHODH related disease or disorder in the subject.

### Intravenous or Subcutaneous Delivery

A pharmaceutical composition having applications in a method of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLETT™, and OPTICLIK™ (sanofi-aventis, Frankfurt, Germany), to name only a few. Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly).

A pharmaceutical composition of the present invention can be also delivered in a vesicle, in particular a liposome (see Langer, 1990, Science 249:1527-1533; Treat et al., 1989, in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, a pharmaceutical composition of the present invention can be delivered in a controlled release system. In one embodiment, a pump may be used (see Langer, supra; Sefton, 1987, CRC Crit. Ref. Biomed. Eng. 14:201). In another embodiment, polymeric materials can be used; see Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying a compound as described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

The following example is presented in order to more fully illustrate the invention. It should in no way be construed, however, as limiting the broad scope of the invention.

### Example

In a search of novel inhibitors for human DHODH, virtual screening compounds was performed, and selected compounds were subsequently tested in a human DHODH enzymatic assay.³⁶ Also determined was the binding mode of selected hits by X-ray crystallography. Provided herein are amino-benzoic acids as novel inhibitors of hDHODH discovered by virtual screening and confirmed by experimental studies

The software program GLIDE was used for virtual screening with the initial vHTS setting followed by the SP procedure.³⁷ Docking was carried out with constraints by including one H-bonding interaction with the side chain of either Arg-136 or Tyr-265 and a hydrophobic pharmacophore feature 6-8 Å away from the hydrogen bond. Selected compounds were tested in the human DHODH enzymatic assay, which resulted in hits with IC₅₀<10 μM (in comparison to an IC₅₀ of 3 nM for brequinar) of which four are shown in Table 1.

TABLE 1
Amino-benzoic Acid Inhibitors for the hDHODH

| | Com- | IC₅₀ |
|---|---|---|
| Structure | pound | (μM) |
| 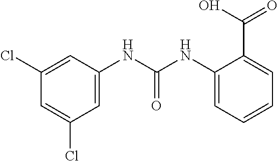 | 1 | 0.7 |
| 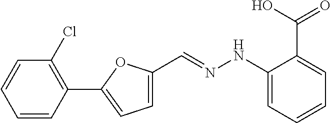 | 2 | 0.5 |
| 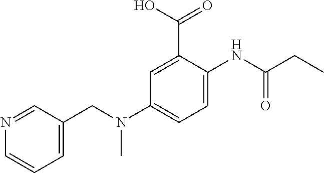 | 3 | <5.5 |
| 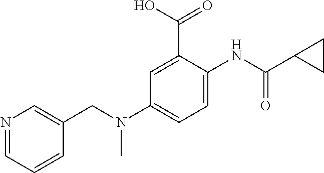 | 4 | <0.1 |

The binding modes of the compounds were confirmed by co-crystal structures with hDHODH, determined by X-ray crystallography. The compounds are found at the coenzyme Q site, as expected. L-dihydroorotate (DHO), flavin mononucleotide (FMN), and the detergent C1 1DAO are also observed in the structures which were solved at a resolution of 1.9-2.1 Å.³⁸ The inhibitor electron densities are well-resolved in all four complexes. The overall position and the orientation of the acid group toward Arg-136 is similar to that previously observed for brequinar in human DHODH (1 D3 G).³²

For compound 1, a hydrogen-bond network is observed around the acidic group that makes bifurcated hydrogen bonds to the guanidinyl group of Arg-136. One carboxylate oxygen atom engages a hydrogen-bond to a crystallographic water molecule, which hydrogen-bonds to Gln-47. The other carboxylate oxygen involves a hydrogen-bond to another crystallographic water, which in turn hydrogen-bonds to the NHs of the urea linker of the inhibitor and the main chain carbonyl oxygen of Thr-360.

The acidic phenyl ring lies in a hydrophobic pocket bounded by the flavin mononucleotide and the side-chains of Val-134 and Tyr-356, limiting potential substitutions on the ring that could be modified to increase potency. In contrast, the dichlorophenyl ring points toward the aqueous phase and offers several opportunities for structural modification to improve the physical properties of the compounds.

A similar hydrogen-bond network is observed around the acidic group of inhibitor 2, except that the second crystallographic water is hydrogen bound to the orthoNH instead of the urea NHs of 1. Compared with 1 (in gold), inhibitor 2 (in white) extends further toward the solvent, as a result of the interposition of a furan ring. The aromatic chlorophenyl group may be favored in this position, due to its proximity to the side-chains of Leu42, Tyr-38, Leu-68, and Phe-62, which form a hydrophobic pocket near the entrance to the ligand binding site. The central region of the inhibitor and the furan ring do not form any close-range interactions with the protein. The chlorine of the phenyl ring is buried and 3.0 Å from the side-chain hydroxyl of Thr-63. The para position of the chlorophenyl is fully exposed to the solvent.

The structural identification of inhibitors 3 and 4 was ultimately revealed by X-ray crystallographic structures. Initially, both 3 and 4 were thought to be the corresponding alkyl amide derivatives, which were actually the structures used for virtual screening. The actual amides were re-synthesized only to show weak inhibition in the hDHODH enzymatic assay, suggesting that the activities of the previous samples may be attributed to impurities or hydrolyzed products or reactants. Indeed, the N-alkyl group is not seen in the original omit map prior to positioning the inhibitor, nor can it be found in the final maps. Rather, the electron density maps are consistent with hydrolysis of the amide to the acid. Once the scaffold was clearly defined, compound 3 was fitted within the electron density, leaving no space for the N-alkyl group to fit in. The final electron density map is fully consistent with the structure of 3, including the conformations around the ethyl-amide and the N-linked methyl-pyridine.

Both carboxylate oxygens hydrogen bond to the side-chain of Arg-136; one oxygen hydrogen bonds to Gln-47 and the second to a crystallographic water. The carbonyl oxygen of the ethylamide is hydrogen-bonded to Tyr-356 hydroxyl. The corresponding NH forms an internal hydrogen-bond to the carboxylate oxygen (2.5 Å).

The crystallographic water molecule near the side-chain of Arg-136 in 3 is in the same position as that observed in 1 and 2. The acidic group of 3 adopts a different orientation relative to the Arg side-chain, as can be seen in a comparison with 2 (in white). Thus, the second crystallographic water is displaced by one of the oxygen atoms of the acidic group in 3. This altered orientation appears to be the result of steric constraints that prevent the amido-ethyl group from burying more deeply into the protein, thus pushing the carboxylate away from the position it adopts in the benzoic acids. The end result is that the acidic group is not as well-disposed for interactions with the side-chain of Arg-136 as the corresponding group in brequinar or other related compounds.

The resulting structure of 4 is the same as that of 3 (including the lengths of the hydrogen-bonds), except for the amido-cyclopropyl in place of amido-ethyl. The cyclopropyl ring lies in a hydrophobic pocket and points toward Val-134. It appears that the pyridine interactions with Thr-63, the amide oxygen hydrogen-bond to Tyr-356, and the near perfect fit of the cyclo-propyl ring in a hydrophobic pocket more than make up for the suboptimal orientation of the carboxylate, resulting in the high activity of compound 4.

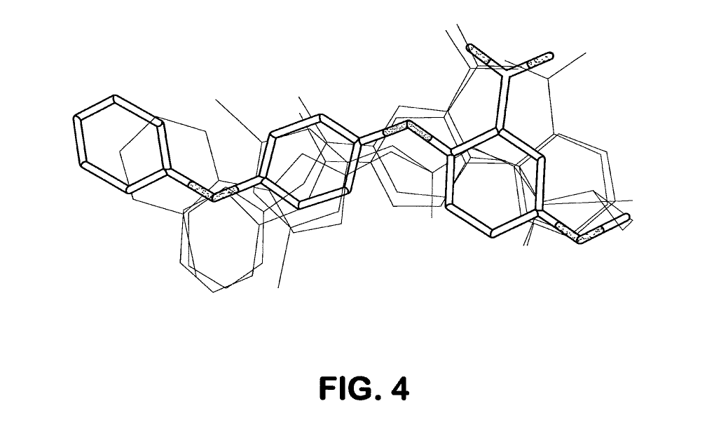

Expanding the repertoire of carboxylic acid inhibitors for the DHODH, meta- and meta-ortho-substituted benzoic acids were discovered by structure-based virtual screening and X-ray structures. FIG. 4 shows the superposition of compounds 1-4 with a recently reported amino-benzoic acid inhibitor in complex with hDHODH (2PRL, with IC₅₀ 81 nM, similar to our compound 4).³⁴ These data demonstrate that the acidic group can span a range of orientations towards the Arg136. This orientation appears to be related to the size of the group that buries in the pocket. The acidic group of compounds 1 and 2 is most deeply buried, as no steric constraint of a second substitution on the ring is present. The corresponding group in compounds 3 and 4 is pushed away from this buried position by the relatively large groups in the ortho position. 2PRL has a substitution of intermediate length, which results in the orientation lying between those reported for the current sanofi-aventis compounds.

In addition, although all of the amino-benzoic acid scaffolds occupy the same binding pocket, subtle changes (such as ethyl to cyclopropyl) can have dramatic effects on inhibitory activities. The dramatic effect of the cyclopropyl group was attributed to an accessible bisected conformation,³⁸ but it might also have resulted from a snug hydrophobic packing of the cyclopropyl group that improved the affinity. The structure-activity information gained from these inhibitors will help further studies of finding a DHODH inhibitor for the development of potential therapeutics for cancer and immunological disorders, examples of which are discussed above.

The following is a list of documents related to the above disclosure and particularly to the experimental procedures and discussions.
1. Evans, D. R.; Guy, H. I., Mammalian pyrimidine biosynthesis: fresh insights into an ancient pathway. J Biol Chem 2004, 279, (32), 33035-8.
2. Loffler, M.; Grein, K.; Knecht, W.; Klein, A.; Bergjohann, U., Dihydroorotate dehydrogenase. Profile of a novel target for antiproliferative and immunosuppressive drugs. Adv Exp Med Biol 1998, 431, 507-13.
3. Baumann, P.; Mandl-Weber, S.; Volkl, A.; Adam, C.; Bumeder, I.; Oduncu, F.; Schmidmaier, R., Dihydroorotate dehydrogenase inhibitor A77 1726 (leflunomide) induces apoptosis and diminishes proliferation of multiple myeloma cells. Mol Cancer Ther 2009, 8, (2), 366-75.
4. Chen, S. F.; Ruben, R. L.; Dexter, D. L., Mechanism of action of the novel anticancer agent 6-fluoro-2-(2′-fluoro-1,1′-biphenyl-4-yl)-3-methyl-4-quinolinecarboxylic acid sodium salt (NSC 368390): inhibition of de novo pyrimidine nucleotide biosynthesis. Cancer Res 1986, 46, (10), 5014-9.
5. Herrmann, M. L.; Schleyerbach, R.; Kirschbaum, B. J., Leflunomide: an immunomodulatory drug for the treatment of rheumatoid arthritis and other autoimmune diseases. Immunopharmacology 2000, 47, (2-3), 273-89.
6. Merrill, J. E.; Hanak, S.; Pu, S. F.; Liang, J.; Dang, C.; Iglesias-Bregna, D.; Harvey, B.; Zhu, B.; McMonagleStrucko, K., Teriflunomide reduces behavioral, electrophysiological, and histopathological deficits in the Dark Agouti rat model of experimental autoimmune encephalomyelitis. J Neurol 2009, 256, (1), 89-103.
7. Warnke, C.; Meyer Zu Horste, G.; Hartung, H. P.; Stuve, O.; Kieseier, B. C., Review of teriflunomide and its potential in the treatment of multiple sclerosis. Neuropsychiatr Dis Treat 2009, 5, 333-40.
8. O'Connor, P. W.; Li, D.; Freedman, M. S.; Bar-Or, A.; Rice, G. P.; Confavreux, C.; Paty, D. W.; Stewart, J. A.; Scheyer, R., A Phase II study of the safety and efficacy of teriflunomide in multiple sclerosis with relapses. Neurology 2006, 66, (6), 894-900.
9. Gero, A. M.; O'Sullivan, W. J., Purines and pyrimidines in malarial parasites. Blood Cells 1990, 16, (2-3), 467-84; discussion 485-98.
10. Subbayya, I. N.; Ray, S. S.; Balaram, P.; Balaram, H., Metabolic enzymes as potential drug targets in Plasmodium falciparum. Indian J Med Res 1997, 106, 79-94.
11. Arakaki, T. L.; Buckner, F. S.; Gillespie, J. R.; Malmquist, N. A.; Phillips, M. A.; Kalyuzhniy, O.; Luft, J. R.; Detitta, G. T.; Verlinde, C. L.; Van Voorhis, W. C.; Hol, W. G.; Merritt, E. A., Characterization of Trypanosoma brucei dihydroorotate dehydrogenase as a possible drug target; structural, kinetic and RNAi studies. Mol Microbiol 2008, 68, (1), 37-50.
12. DeFrees, S. A.; Sawick, D. P.; Cunningham, B.; Heinstein, P. F.; Morre, D. J.; Cassady, J. M., Structure-activity relationships of pyrimidines as dihydroorotate dehydrogenase inhibitors. Biochem Pharmacol 1988, 37, (20), 3807-16.
13. Davies, M.; Heikkila, T.; McConkey, G. A.; Fishwick, C. W.; Parsons, M. R.; Johnson, A. P., Structure-based design, synthesis, and characterization of inhibitors of human and Plasmodium falciparum dihydroorotate dehydrogenases. J Med Chem 2009, 52, (9), 2683-93.
14. Baumgartner, R.; Walloschek, M.; Kralik, M.; Gotschlich, A.; Tasler, S.; Mies, J.; Leban, J., Dual binding mode of a novel series of DHODH inhibitors. J Med Chem 2006, 49, (4), 1239-47.
15. Hurt, D. E.; Sutton, A. E.; Clardy, J., Brequinar derivatives and species-specific drug design for dihydroorotate dehydrogenase. Bioorg Med Chem Lett 2006, 16, (6), 1610-5.
16. Hansen, M.; Le Nours, J.; Johansson, E.; Antal, T.; Ullrich, A.; Loffler, M.; Larsen, S., Inhibitor binding in a class 2 dihydroorotate dehydrogenase causes variations in the membrane-associated N-terminal domain. Protein Sci 2004, 13, (4), 1031-42.
17. Deng, X.; Gujjar, R.; El Mazouni, F.; Kaminsky, W.; Malmquist, N. A.; Goldsmith, E. J.; Rathod, P. K.; Phillips, M. A., Structural plasticity of malaria dihydroorotate dehydrogenase allows selective binding of diverse chemical scaffolds. J Biol Chem 2009.
18. Gujjar, R.; Marwaha, A.; El Mazouni, F.; White, J.; White, K. L.; Creason, S.; Shackleford, D. M.; Baldwin, J.; Charman, W. N.; Buckner, F. S.; Charman, S.; Rathod, P. K.; Phillips, M. A., Identification of a metabolically stable triazolopyrimidine-based dihydroorotate dehydrogenase inhibitor with antimalarial activity in mice. J Med Chem 2009, 52, (7), 1864-72.
19. Patel, V.; Booker, M.; Kramer, M.; Ross, L.; Celatka, C. A.; Kennedy, L. M.; Dvorin, J. D.; Duraisingh, M. T.; Sliz, P.; Wirth, D. F.; Clardy, J., Identification and characterization of small molecule inhibitors of Plasmodium falciparum dihydroorotate dehydrogenase. J Biol Chem 2008, 283, (50), 35078-85.
20. Phillips, M. A.; Gujjar, R.; Malmquist, N. A.; White, J.; El Mazouni, F.; Baldwin, J.; Rathod, P. K., Triazolopyrimidine-based dihydroorotate dehydrogenase inhibitors with potent and selective activity against the malaria parasite Plasmodium falciparum. J Med Chem 2008, 51, (12), 3649-53.
21. Deguchi, M.; Kishino, J.; Hattori, M.; Fume, Y.; Yamamoto, M.; Mochizuki, I.; Iguchi, M.; Hirano, Y.; Hojou, K.; Nagira, M.; Nishitani, Y.; Okazaki, K.; Yasui, K.; Arimura, A., Suppression of immunoglobulin production by a novel dihydroorotate dehydrogenase inhibitor, S-2678. Eur J Pharmacol 2008, 601, (1-3), 163-70.
22. Heikkila, T.; Ramsey, C.; Davies, M.; Galtier, C.; Stead, A. M.; Johnson, A. P.; Fishwick, C. W.; Boa, A. N.; McConkey, G. A., Design and synthesis of potent inhibitors of the malaria parasite dihydroorotate dehydrogenase. J Med Chem 2007, 50, (2), 186-91.
23. Heikkila, T.; Thirumalairajan, S.; Davies, M.; Parsons, M. R.; McConkey, A. G.; Fishwick, C. W.; Johnson, A. P., The first de novo designed inhibitors of Plasmodium falciparum dihydroorotate dehydrogenase. Bioorg Med Chem Lett 2006, 16, (1), 88-92.
24. Herz, T.; Wolf, K.; Kraus, J.; Kramer, B., 4SCan/vADME: intelligent library screening as a shortcut from hits to lead compounds. Expert Opin Drug Metab Toxicol 2006, 2, (3), 471-84.
25. Leban, J.; Kralik, M.; Mies, J.; Baumgartner, R.; Gassen, M.; Taster, S., Biphenyl-4-ylcarbamoyl thiophene carboxylic acids as potent DHODH inhibitors. Bioorg Med Chem Lett 2006, 16, (2), 267-70.
26. Boa, A. N.; Canavan, S. P.; Hirst, P. R.; Ramsey, C.; Stead, A. M.; McConkey, G. A., Synthesis of brequinar analogue inhibitors of malaria parasite dihydroorotate dehydrogenase. Bioorg Med Chem 2005, 13, (6), 1945-67.
27. Baldwin, J.; Michnoff, C. H.; Malmquist, N. A.; White, J.; Roth, M. G.; Rathod, P. K.; Phillips, M. A., High-throughput screening for potent and selective inhibitors of Plasmodium falciparum dihydroorotate dehydrogenase. J Biol Chem 2005, 280, (23), 21847-53.
28. Kobayashi, K.; Nakashima, A.; Nagata, H.; Nakajima, H.; Yamaguchi, K.; Sato, S.; Mild, I., Anti-arthritic effects of KF20444, a new immunosuppressive compound inhibiting dihydroorotate dehydrogenase, on rat collagen-induced arthritis. Inflamm Res 2001, 50, (1), 24-31.
29. Papageorgiou, C.; von Matt, A.; Joergensen, J.; Andersen, E.; Wagner, K.; Beerli, C.; Than, T.; Borer, X.; Florineth, A.; Rihs, G.; Schreier, M. H.; Weckbecker, G.; Heusser, C., Aromatic quinolinecarboxamides as selective, orally active antibody production inhibitors for prevention of acute xenograft rejection. J Med Chem 2001, 44, (12), 1986-92.
30. Papageorgiou, C.; Albert, R.; Floersheim, P.; Lemaire, M.; Bitch, F.; Weber, H. P.; Andersen, E.; Hungerford, V.; Schreier, M. H., Pyrazole bioisosteres of leflunomide as B-cell immunosuppressants for xenotransplantation and chronic rejection: scope and limitations. J Med Chem 1998, 41, (18), 3530-8.
31. Kuo, E. A.; Hambleton, P. T.; Kay, D. P.; Evans, P. L.; Matharu, S. S.; Little, E.; McDowall, N.; Jones, C. B.; Hedgecock, C. J.; Yea, C. M.; Chan, A. W.; Hairsine, P. W.; Ager, I. R.; Tully, W. R.; Williamson, R. A.; Westwood, R., Synthesis, structure-activity relationships, and pharmacokinetic properties of dihydroorotate dehydrogenase inhibitors: 2-cyano-3-cyclopropyl-3-hydroxy-N-[3′-methyl-4′-(trifluoromethyl)phenyl]propenamide and related compounds. J Med Chem 1996, 39, (23), 4608-21.
32. Liu, S.; Neidhardt, E. A.; Grossman, T. H.; Ocain, T.; Clardy, J., Structures of human dihydroorotate dehydrogenase in complex with antiproliferative agents. Structure 2000, 8, (1), 25-33.
33. Hurt, D. E.; Widom, J.; Clardy, J., Structure of Plasmodium falciparum dihydroorotate dehydrogenase with a bound inhibitor. Acta Crystallogr D Biol Crystallogr 2006, 62, (Pt 3), 312-23.
34. Walse, B.; Dufe, V. T.; Svensson, B.; Fritzson, I.; Dahlberg, L.; Khairoullina, A.; Wellmar, U.; AlKaradaghi, S., The structures of human dihydroorotate dehydrogenase with and without inhibitor reveal conformational flexibility in the inhibitor and substrate binding sites. Biochemistry 2008, 47, (34), 8929-36.
35. McLean, J. E.; Neidhardt, E. A.; Grossman, T. H.; Hedstrom, L., Multiple inhibitor analysis of the brequinar and leflunomide binding sites on human dihydroorotate dehydrogenase. Biochemistry 2001, 40, (7), 2194-200.
36. Assay, conditions as described in reference 16.
37. Schrodinger, I., New York, N.Y., USA (www.schrodinger.com).
38. Kuo, P. Y.; Shie, T. L.; Chen, Y. S.; Lai, J. T.; Yang, D. Y., Enzyme inhibition potency enhancement by active site metal chelating and hydrogen bonding induced conformation-restricted cyclopropanecarbonyl derivatives. Bioorg Med Chem Lett 2006, 16, (23), 6024-7.

Many other variations and modifications of the invention will be apparent to those skilled in the art without departing from the scope of the invention. The above-described descriptions are, therefore, intended to be merely exemplary, and all such variations and modifications are intended to be included within the scope of the invention as defined in the appended claims.

What is claimed is:

1. A method for decreasing dihydroorotate dehydrogenase (DHODH) activity, comprising contacting the DHODH with a compound: 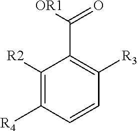
wherein:
R₁ is H, an alkyl or lower alkyl group, or a halogen;
R₂ is: 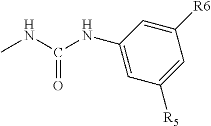
wherein R₅ is a halogen and R₆ is a halogen, 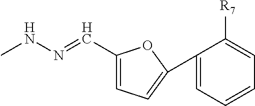
wherein R₇ is a halogen, or
H;
R₃ is:
H, 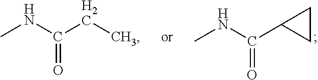
and
R₄ is:
H, or 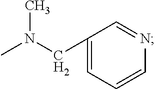
an N-oxide of the compound, a prodrug of the compound, a pharmaceutically acceptable salt of the compound, a solvate of the compound, or a hydrate of the compound,
wherein contacting the compound with the DHODH decreases the DHODH activity of the DHODH prior to contacting the compound with the DHODH, or decreases the DHODH activity with respect to the DHODH activity a control DHODH that is not contacted with the compound.

2. The method of claim 1, wherein:
R₁ is H;
R₂ is 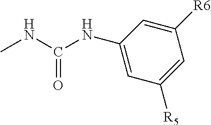
wherein R₅ and R₆ are each a halogen;
R₃ is H; and
R₄ is H.

3. The method of claim 2, wherein R₅ is Cl and R₆ is Cl.

4. The method of claim 3, wherein:
R₁ is H;
R₂ is 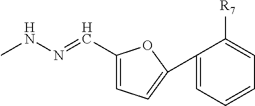
wherein R₇ is Cl;
R₃ is H; and
R₄ is H.

5. The method of claim 1, wherein:
R₁ is H;
R₂ is H;
R₃ is 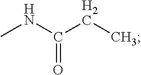
and
R₄ is

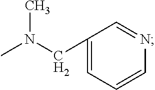

6. The method of claim 1, wherein:
R₁ is H;
R₂ is H;
R₃ is 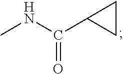
and
R₄ is

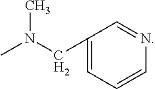

7. A method for decreasing DHODH activity comprising contacting the DHODH with a compound: 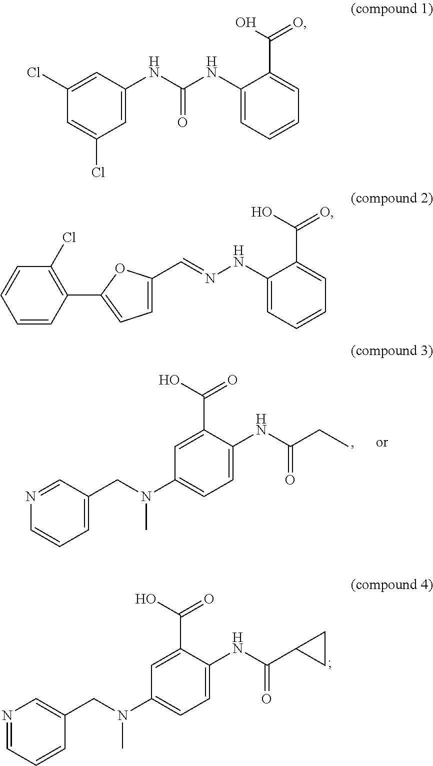
an N-oxide of the compound, a prodrug of the compound, a pharmaceutically acceptable salt of the compound, a solvate of the compound, or a hydrate of the compound,
wherein contacting the compound with the DHODH decreases the DHODH activity with respect to the DHODH activity of the DHODH prior to contacting the compound with the DHODH, or decreases the DHODH activity with respect to the DHODH activity a control DHODH that is not contacted with the compound.

8. A pharmaceutical composition comprising a compound and a pharmaceutically acceptable carrier, wherein the compound is: 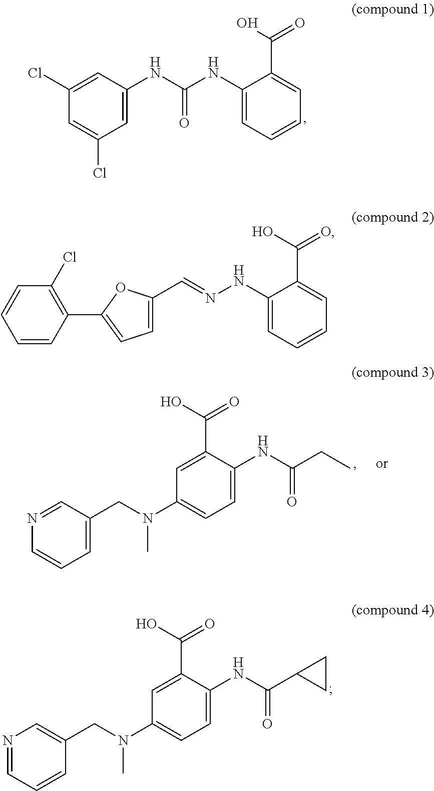
an N-oxide of the compound, a prodrug of the compound, a pharmaceutically acceptable salt of the compound, a solvate of the compound, or a hydrate of the compound.

9. A method of treating a DHODH related disease or disorder in a subject, comprising administering to the subject a therapeutically effective amount of a compound: 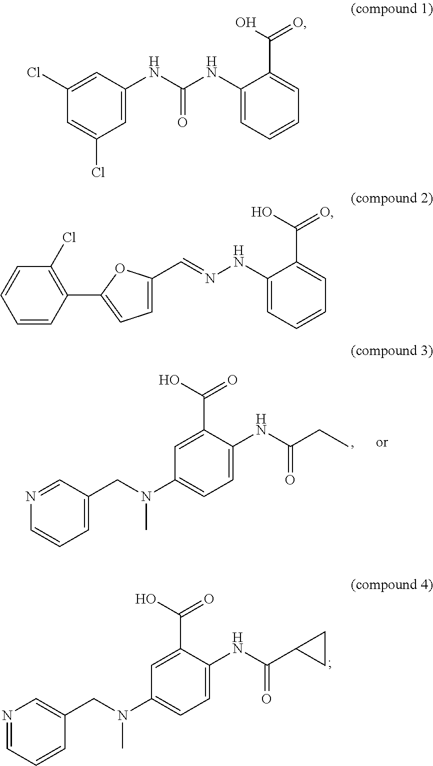
an N-oxide of the compound, a prodrug of the compound, a pharmaceutically acceptable salt of the compound, a solvate of the compound, or a hydrate of the compound.

10. The method of claim 9, wherein the DHODH related disease or disorder is cancer or an immunological disorder.

11. The method of claim 10, wherein the immunological disorder is rheumatoid arthritis and multiple sclerosis.
